(19) United States
(12) Patent Application Publication
(10) Pub. No.: US 2016/0192901 A1
(43) Pub. Date: Jul. 7, 2016

(54) WARMING GEL SYSTEM AND METHOD

(71) Applicant: THERMAL-GEL, LLC, Hewitt, TX (US)

(72) Inventors: Edward Patrick O'Boyle, JR., Hewitt, TX (US); Xiaojiang Wang, Ann Arbor, MI (US); Evan Boyst, Ann Arbor, MI (US)

(21) Appl. No.: 14/591,907

(22) Filed: Jan. 7, 2015

(51) Int. Cl. A61B 8/00; A61K 49/22

(52) U.S. Cl. CPC A61B 8/4281; A61K 49/22

(57) ABSTRACT

A method of applying ultrasound gel including the steps of removing a barrier separating a reagent and a lubricating gel. The reagent and the lubricating gel are separated within a single packaging. Mixing the reagent and the lubricating gel to produce a lubricating gel mixture. The mixing causes an exothermic reaction configured to selectively heat the lubricating gel to a desired temperature. Opening the packaging to permit the release of the lubricating gel mixture and then distributing the lubricating gel mixture onto a patient.

### BACKGROUND

1. Field of the Invention

The present application relates generally to ultrasound procedures and, more particularly, to an improved ultrasound gel and method of applying the self-warming gel.

2. Description of Related Art

Ultrasound technologies are used to detect objects and measure distances in both veterinary medicine and human medicine. Ultrasound technologies can be used for medical imaging, detection, and measurement to name a few. Medical sonography (ultrasonography) is an ultrasound-based diagnostic medical imaging technique used to visualize muscles, tendons, and many internal organs, to capture their size, structure and any pathological lesions with real time tomographic images. Ultrasound has been used by radiologists and sonographers to image the human body for decades and is a commonly accepted diagnostic tool. Examples of uses are the measurement and imaging of human fetuses and the evaluation of soft tissue and tendon injuries in horses.

Equipment typically involves the use of a probe (transducer) which emits and receives a sound wave to produce an image. A water-based gel is typically used between the patient and the probe. Conventional gels have many disadvantages, such as an inability to remain in localized contact with the patient without evaporation or decreased viscosity; being cold to the touch; and contamination, to name a few.

To address the cold temperature, it has been known that medical professionals will attempt to warm the gel. Common warming methods include a microwave or warming plate. Such methods frequently result in difficulty obtaining a pleasing temperature, meaning the gel is made either too hot or not hot enough. Even then, the act of warming a gel can cause further contamination. Gels are often left within a container in warmed conditions where contamination issues have been known to develop.

Furthermore, another disadvantage of conventional gel is the container itself. Containers fail to provide a premeasured amount of gel for each use. This leads to operators applying varied amounts of gel for the procedure. A too minimal amount requires additional applications of gel while too excessive amounts yield waste. Additionally, the lack of viscosity and evaporation of the gel can necessitate multiple applications of gel during a single imaging process.

These and other disadvantages remain with conventional ultrasound gels and the method of storing/using them. It is desirable to provide an improved ultrasound gel and method of use. Considerable shortcomings remain.

### DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is an exemplary top view of a gel warming system according to the preferred embodiment of the present application;

FIG. 2 is a table of ingredients used within a gel of the gel warming system of FIG. 1;

FIG. 3 is a table of ingredients of an exemplary embodiment of the gel of FIG. 2;

FIG. 4 is a chart of the process of making the gel of FIG. 3;

FIG. 5 is a table of ingredients of a second exemplary embodiment of the gel of FIG. 2;

FIG. 6 is a chart of the process of making the gel of FIG. 5; and

FIG. 7 is a chart of the method of using the gel of FIG. 2.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the device described herein may be oriented in any desired direction.

The system and method of use in accordance with the present application overcomes one or more of the above-discussed problems commonly associated with existing ultrasound lubricating gels. Specifically, the system and method of the present application is configured to provide a single-use sealed package containing a portion for the gel and a portion for a reagent, wherein the reagent and the gel are selectively mixed to generate a controlled exothermic reaction used to warm the gel to a desired temperature. The gel may then be removed from the package and applied to the patient for use. The gel is configured to adhere to the skin of the patient and retain greater viscosity as a result of its chemical composition. The gel remains sealed and uncontaminated, opened only upon use. These and other unique features of the apparatus are discussed below and illustrated in the accompanying drawings.

The system and method will be understood, both as to its structure and operation, from the accompanying drawings, taken in conjunction with the accompanying description. Several embodiments of the system are contemplated herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the figures wherein like reference characters identify corresponding or similar elements in form and function throughout the several views. The following Figures describe the system and method of the warming lubricating gel and its associated features to provide improved safety, comfort of the patient, and utilization of resources by minimizing waste. It is understood that the principles described herein are applicable to other types of gels besides those of ultrasound lubricating gels. Therefore, the features associated with the gel of the present application are adaptable and possible to associate with other gels. Therefore these features are used merely for exemplary purposes in the description, but in no wise act to limit the present application to only ultrasound lubricating gel.

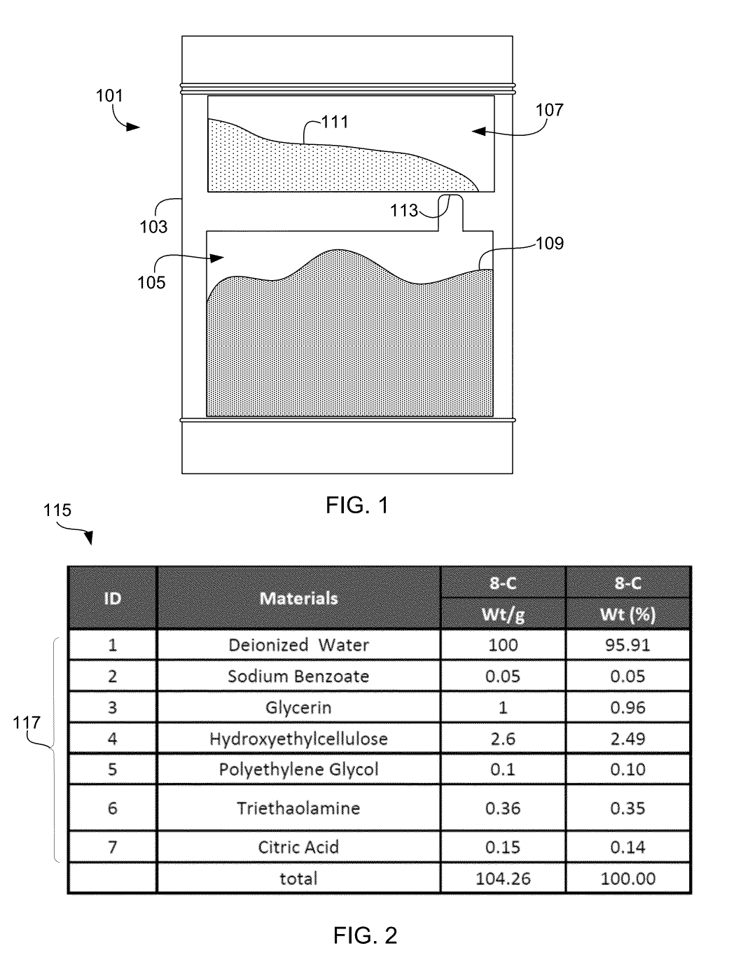

Referring now to FIG. 1 in the drawings, a system for providing a warmed lubricating gel to a patient is illustrated. System 101 includes a package 103 having a gel portion 105 and a reagent portion 107. Gel portion 105 is a sealed unit configured to hold a lubricating gel 109 for use with ultrasound technologies. Reagent portion 107 is a sealed unit configured to hold a reagent 111 configured to warm gel 109. Gel 109 and reagent 111 are included within system 101. As illustrated in FIG. 1, packaging 103 includes portions 105 and 107.

System 101 is configured to provide a sealed and sterile method of storing, transporting, heating, and using gel 109 for use with ultrasound technologies to address one or more of the issued described above with respect to conventional gels and techniques. Packaging 103 is a sealed container having respective portions 105 and 107 for containing gel 109 and reagent 111. Prior to use, packaging 103 is sealed from the elements, thereby maintaining sanitation standards for a sterile environment and avoiding contamination of gel 109. At the time of use, gel 109 and reagent 111 are selectively mixed, producing an exothermic reaction that warms the gel. The mixing of gel 109 and reagent 111 is configured to be maintained within packaging 103, thereby maintaining the sterile feature. Only upon opening packaging 103 for application of gel 109 to the skin of a patient is gel 109 exposed to the external elements.

Packaging 103 is configured to separately locate gel 109 and reagent 111 and to permit the selective mixing of such items. As illustrated in FIG. 1, packaging 103 is exemplified as a flexible bag or pouch wherein portions 105 and 107 are integrally formed within packaging 103. It is preferred that packaging 103 is transparent to permit visualization of gel 109 and reagent 111, but it is not limited to being such. Other coloring options or graphic indicia are contemplated for use with packaging 103.

Gel portion 105 is located below reagent portion 107 and are separated from one another by a barrier 113. Barrier 113 is configured to separate gel portion 105 from reagent portion 107, so as to selectively regulate the mixture of lubricating gel 109 and reagent 111. Barrier 113 is a relatively thin portion of packaging 103 that is sealed together. To open barrier 113 and permit mixing of gel 109 and reagent 111, a pressure is applied to packaging 103 sufficient to increase the internal pressure within portion 105 and/or portion 107 sufficient to remove or open barrier 113. By removal of barrier 113, it is understood that the seal of packaging 103 is broken.

Although packaging 101 has been described as such above, it is understood that packaging 103 may take other forms and features, such that portions of packaging 103 may not be flexible in other embodiments. Additionally, barrier 113 may be any other type of divider that adequately separates two or more connected regions. Barrier 113 may be a separate item from that of packaging 103 in other embodiments. Additionally, barrier 113 may be opened in many different ways in other embodiments, such as: by removal of itself from packaging 103, by puncturing, stretching, and so forth. It is important to note that packaging 103 is configured to separately locate gel 109 from that of reagent 111 and system 101 as a whole is configured to permit their selective mixing prior to use.

Some key features of system 101 are as follows: System 101 is configured to minimize waste. The amount of gel 109 contained within gel portion 105 is premeasured and is of sufficient quantity to perform an ultrasound. The precise amount may be pre-selected based upon the type of procedure performed on the patient. An operator is not required to arbitrarily apply an amount of gel. By maintaining the gel 109 in prepackaged units, tracking the amount of gel 109 used and inventorying the amount left becomes much simpler.

An additional advantage of system 101 is that the packaging is designed for a single-use, meaning that the packaging 103 (or portion of packaging 103) is to be disposed of once opened. Although reusable packaging is contemplated, it is understood that an advantage of system 101 is that by not mixing existing gel with newer gels the risks associated with contamination are greatly reduced. Therefore packaging 103 is configured to separately locate gel portion 105 from reagent portion 107. However, portions of the packaging may be retained while portions in contact with gel 109 and/or reagent 107 may be disposed of. For example, wherein portions 105 and 107 are not integrally formed within packaging 103, each portion 105, 107 may be selectively disposed of after use.

Referring now also to FIG. 2 in the drawings, a table 115 of ingredients used within gel 109 is illustrated. Gel 109 is composed of a plurality of ingredients. FIG. 2 illustrates some key ingredients that operate to form a base mixture 117 for gel 109. Gel 109 includes a nonionic thickener mixture that is composed largely of water. It is understood that various types of nonionic gels may be formed from base mixture 117 by incorporating differing amounts of ingredients. Additional thickeners beside the one used in mixture 117 may be polyurethane dispersions, crystalline hydrated magnesium alumino-silicate, and other associative mineral and polymer thickeners for example. Reagent 111 is an anhydrous calcium chloride powder but can be other types of powder, such as: metal halide (i.e. MgCl₂) and metal oxide (i.e. ZnO, CaO, and MgO).

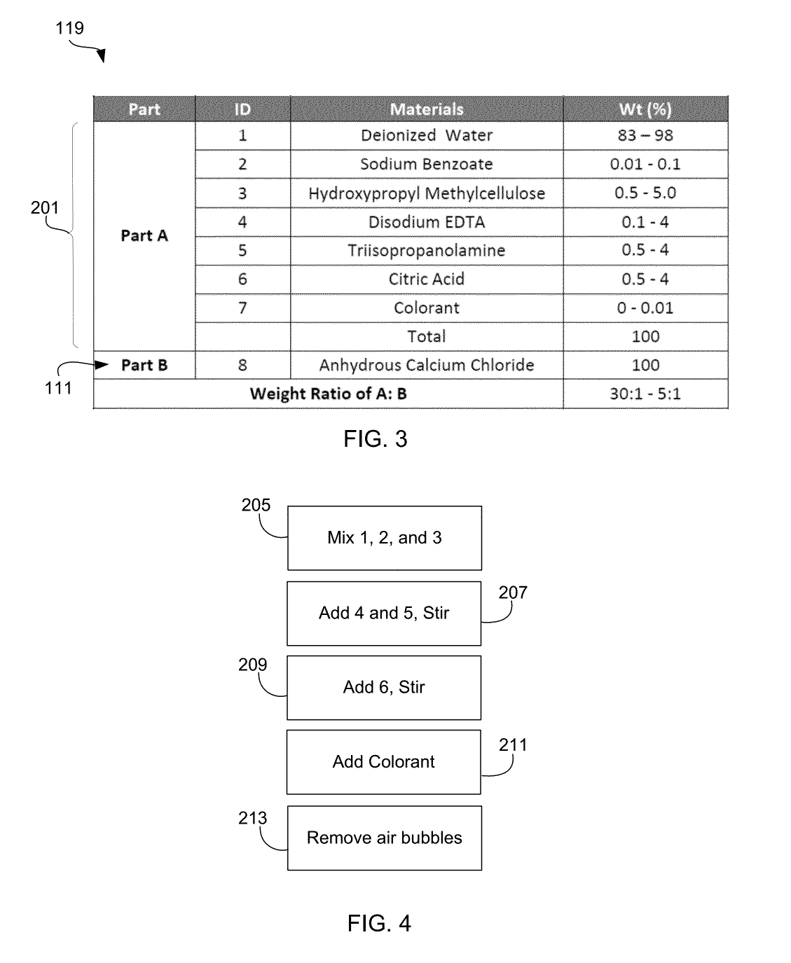

Referring now also to FIGS. 3 and 4 in the drawings, a table 119 of ingredients for an exemplary gel 201 is illustrated along with steps to formulate the gel mixture. Gel 201 is similar in form and function to that of gel 109, however as seen in table 119, some of the specific ingredients of gel 201 differ from that of gel 109. Gel 201 is a hydroxypropyl methylcellulose based self-warming ultrasound gel formulation. Gel 201 is selectively mixed with reagent 111 to produce an exothermic reaction to self-warm gel 201 to a predetermined temperature. The exact temperature is regulated by the ratio of reagent to gel. The combination of gel 201 and reagent 111 form a gel mixture.

The procedure for making gel 201 includes a number of steps. First, ingredients 1, 2, and 3 are mixed together 205 for approximately five (5) minutes at room temperature. Ingredients 4 and 5 are added 207 and stirred into the mixture for approximately twenty (20) min, sufficient to obtain a clear gel substance. Ingredient 6 is added 209 to the mixture and stirred approximately for an additional ten (10) minutes. Next, a suitable colorant is optionally added 211 and stirred approximately for an additional ten (10) minutes. The gel mixture is then processed 213 to remove existing air bubbles. Removal may be performed via a centrifuge process or under vacuum.

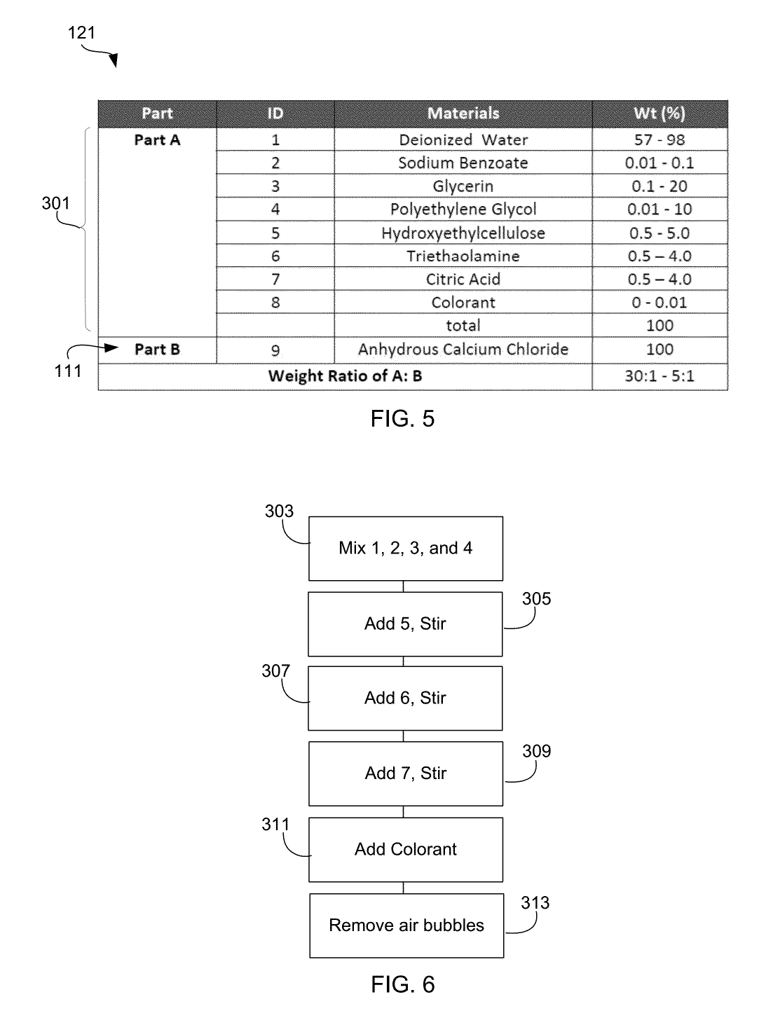

Referring now also to FIGS. 5 and 6 in the drawings, a table 121 of ingredients for an exemplary gel 301 is illustrated along with steps to formulate the gel mixture. Gel 301 is similar in form and function to that of gel 109, however as seen in table 121, some of the specific ingredients of gel 201 differ from that of gel 109. Gel 301 is a hydroxyethylcellulose based self-warming ultrasound gel formulation. Gel 301 is selectively mixed with reagent 111 to produce an exothermic reaction to self-warm gel 201 to a predetermined temperature. The exact temperature is regulated by the ratio of reagent to gel. The combination of gel 201 and reagent 111 form a gel mixture.

The procedure for making gel 301 includes a number of steps, similar to those associated with gel 201. Ingredients 1, 2, 3, and 4 are mixed together 303 at room temperature for approximately ten (10) minutes. Ingredient 5 is added 305 and stirred into the mixture for approximately ten (10) min, sufficient to obtain an equal dispersion within the mixture. Ingredient 6 is added 307 to the mixture and stirred approximately for an additional 20 (20) minutes to obtain a gel. Next, ingredient 7 is added 309 and stirred for approximately ten (10) minutes. A suitable colorant is optionally added 311 and stirred approximately for an additional ten (10) minutes. The gel mixture is then processed 313 to remove existing air bubbles. Removal may be performed via a centrifuge process as done similarly with gel 201.

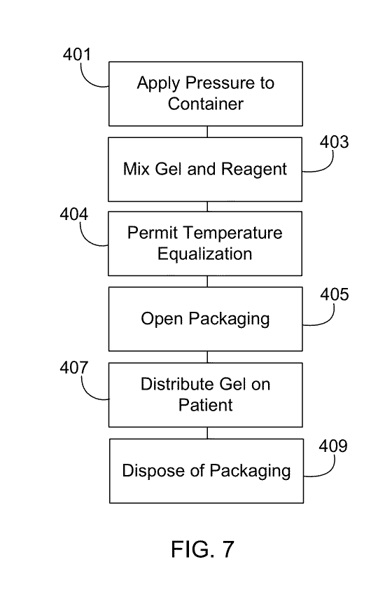

Referring now also to FIG. 7 in the drawings, a chart of the method of using and applying system 101 is illustrated. The steps of using system 101 involve at least the following: removing the barrier 401, mixing the gel and reagent 403, opening the packaging 405, and distributing the gel mixture onto the patient 407. Lubricating gel 109 and reagent 111 are individually packaged and stored in sealed portions. The sealed nature of the packaging prevents contamination.

In operation, the barrier is removed by increasing the pressure within gel portion 105 and/or reagent portion 107, in which barrier 113 bursts or ruptures. After which, gel 109 and reagent 111 are mixed together, thereby instigating an exothermic reaction. During the reaction, the temperature of the gel increases. The level or the max temperature the gel reaches is regulated. System 101 is configured to selectively provide a corresponding ratio of reagent 111 to that of gel 109. By adjusting the ratio, the max temperature of the gel can vary. The exothermic reaction is controlled and self contained within sealed packaging 103 therefore fears of contamination of gel 109 during the reaction is minimized. It is important to note that the ultrasonic properties of the lubricating gel are unaffected by the mixing with the reagent and do not negatively affect the transmission of energy.

Temperature levels increase during the mixing process and in time stabilizes. After mixing, the gel mixture may optionally be allotted time 404 to permit temperature equalization within the gel mixture prior to application on the patient. As desired, packaging 103 may be opened 405 to permit the removal or distribution of the gel mixture on the patient. Opening of the packaging may be performed in multiple ways. One example is the tearing of a portion of packaging 103 to form a hole for the gel mixture to evacuate. Another example may be to cut the packaging 103.

Distribution occurs on the area of interest on the patient. The gel 109, 201, 301 is configured to contain and include a thickener to control and stabilize the level of viscosity and to minimize the affect of salts. The viscosity levels are maintained sufficient to prevent runoff of the gel on curved surfaces. Additionally the gel is configured to form a film to decrease friction and allow the transducer/probe the ability to freely transverse the area of interest. Once the gel is distributed, the packaging 103 is disposed of (i.e. discarded, trash). After the procedure is finished, the gel mixture is wiped away and is water soluble.

The current application has many advantages over the prior art including at least the following: (1) Pre-packaged quantity of lubricating gel; (2) Self sealed and sterile packaging; (3) One time use packaging; (4) Self warming lubricating gel; (5) Exothermic reaction occurs in sealed environment; (6) Minimized waste; (7) Controlled temperature level; (8) Environmentally friendly packaging; and (9) Decreased contamination.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A method of applying ultrasound gel, comprising:
removing a barrier separating a reagent and a lubricating gel, wherein the reagent and the lubricating gel are separated within a packaging;
mixing the reagent and the lubricating gel to produce a lubricating gel mixture, the mixing generating an exothermic reaction;
opening the package to permit the release of the lubricating gel mixture; and
distributing the lubricating gel mixture onto a patient.

2. The method of claim 1, wherein the ultrasonic properties of the lubricating gel are unaffected by the mixing with the reagent.

3. The method of claim 1, wherein the barrier is a sealed section of the packaging separating the lubricating gel from the reagent, the barrier configured to selectively open and permit the mixing of lubricating gel and reagent.

4. The method of claim 3, wherein removing the barrier includes increasing the internal pressure within the packaging to a predetermined level so as to permit mixing of the lubricating gel and the reagent.

5. The method of claim 1, wherein the lubricating gel includes a nonionic thickener mixture.

6. The method of claim 1, wherein the packaging is sealed and configured to keep the lubricating gel and reagent sterile until use.

7. The method of claim 1, wherein the packaging is configured for single-use.

8. The method of claim 1, wherein the exothermic reaction occurs within the sealed packaging.

9. The method of claim 1, further comprising:
waiting for the temperature of the lubricating gel to stabilize following mixing with the reagent.

10. The method of claim 1, wherein the volume of lubricating gel mixture within the container is predetermined based upon the procedure performed on the patient.

11. The method of claim 1, further comprising:
disposing of the packaging after distribution of the lubricating gel mixture.

12. The method of claim 1, further comprising:
removing the gel mixture from the patient.

13. The method of claim 1, wherein opening the container is performed by tearing a portion of the container.

14. A gel warming system, comprising:
a sealed package having a gel portion and a reagent portion, the gel portion being separated from the reagent portion;
a reagent located within the reagent portion;
a lubricating gel located within the gel portion; and
a barrier configured to separate the gel portion from the reagent portion, so as to selectively regulate the mixture of lubricating gel and reagent;
wherein the mixture of the lubricating gel and the reagent creates an exothermic reaction that warms the gel to a preselected temperature.

15. The gel warming system of claim 14, wherein the barrier is selectively removed so as to permit mixing of the reagent and the lubricating gel.

16. The gel warming system of claim 14, wherein the mixture of the reagent with the lubricating gel has no adverse affects on the transmission of energy through the lubricating gel.

17. The gel warming system of claim 14, wherein the lubricating gel is heated within the sealed packaging.

18. The gel warming system of claim 14, wherein the gel is nonionic and contains a thickener configured to stabilize the viscosity so as to minimize the affect of salts.

19. The gel warming system of claim 14, wherein the packaging is configured to open and permit the removal of the lubricating gel.
